SEQUENCE LISTING (12 sequences)

SEQ ID NO: 1 - DNA, 218 bp, Homo sapiens
gcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagaga

SEQ ID NO: 2 - DNA, 21 bp, Artificial Sequence
  P3
gcacgttcagaagtcggttaa

SEQ ID NO: 3 - DNA, 22 bp, Artificial Sequence
  P2
tctctagtgttatgttctcgtc

SEQ ID NO: 4 - DNA, 233 bp, Homo sapiens
ctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagaga

SEQ ID NO: 5 - DNA, 20 bp, Artificial Sequence
  P1
ctgtaataggactgcccatc

SEQ ID NO: 6 - DNA, 137 bp, Homo sapiens
gcacgttcagaagtcggttaataacgacatgatagtcactgacaacaacggtgcagtcaagtttccacaactgtgtaaattttgtgatgtgagattttccacctgtgacaaccagaaatcctgcttctccaaagtgc

SEQ ID NO: 7 - DNA, 21 bp, Artificial Sequence
  P4
gcactttggagaagcaggatt

SEQ ID NO: 8 - protein, 167 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQV

SEQ ID NO: 9 - protein, 192 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQV

SEQ ID NO: 10 - protein, 80 aa, Homo sapiens
MGRGLLRGLWPLHIVLWTRIASTIPPHVQKSVNNDMIVTDNNGAVKFPQLCKFCDVRFSTCDNQKSCFSKVHYEGKKKAW

SEQ ID NO: 11 - DNA, 19 bp, Artificial Sequence
  GAPDH-F
gtcagtggtggacctgacc

SEQ ID NO: 12 - DNA, 20 bp, Artificial Sequence
  GAPDH-R
tgagcttgacaaagtggtcg

(19) United States
(12) Patent Application Publication
(10) Pub. No.: US 2019/0391144 A1
(43) Pub. Date: Dec. 26, 2019

(54) METHOD FOR DETERMINING THE ACTIVITY OF AUTOIMMUNE DISEASES AND KIT

(71) Applicants: Benito Jorge VELASCO ZAMORA, Alicante (ES); CONSEJO NACIONAL DE INVESTIGACIONES CIENTÍFICAS Y TÉCNICAS (CONICET), Ciudad Autónoma de Buenos Aires (AR); FUNDACIÓN ARTICULAR, Provincia de Buenos Aires (AR)

(72) Inventors: Benito Jorge VELASCO ZAMORA, Buenos Aires (AR); Ricardo Alfredo DEWEY, Buenos Aires (AR); Alejandra CARREA, Provincia de Buenos Aires (AR); Marcelo Javier Perone, Ciudad Autónoma de Buenos Aires (AR); Matias Adan PREISEGGER, Buenos Aires (AR); Pamela Daiana VAZQUEZ, Buenos Aires (AR)

(73) Assignees: Consejo Nacional de Investigaciones Científicas y Técnicas, Ciudad Autónoma de Buenos Aires (AR); FUNDACIÓN ARTICULAR, Provincia de Buenos Aires (AR); Benito Jorge VELASCO ZAMORA, Buenos Aires (AR)

(21) Appl. No.: 16/349,381

(22) PCT Filed: Nov. 14, 2017
(86) PCT No.: PCT/ES2017/070752

§ 371 Date: May 13, 2019

(30) Foreign Application Priority Data
Nov. 14, 2016 (AR) 20160103481

(51) Int. Cl. G01N 33/564; G01N 1/28

(52) U.S. Cl. CPC G01N 33/564; G01N 21/763; G01N 2333/495; G01N 1/28

(57) ABSTRACT

A method to determine the activity level of autoimmune diseases, the method including a) to supply a biological sample; b) to determine the TßRII-A, TßRII-B and TßRII-Se isoforms level of the biological sample; c) calculate the activity level by performing the quotient between the TßRII-Se level and the level of the addition of TßRII-A y TßRII-B. The isoforms level are measured by detecting the polypeptides of the isoforms or the mRNA of the isoforms in the isolated circulating mononuclear blood cells by means of, for instance, RT-qPCR. The ΔCt of each splice variants individually showed a correlation with autoimmune disease activity. Additionally, a similar correlation with autoimmune disease activity was obtained when the ΔCt of TβRII-SE was added to the ΔCt of TβRII-A and the ΔCt of TβRII-A.

### CROSS REFERENCE TO RELATED APPLICATION

This application is a national stage entry of PCT/ES2017/070752 filed Nov. 14, 2017, under the International Convention and claiming priority over Argentinean application No. 20160103481 filed Nov. 14, 2016.

### FIELD OF THE ART

The present invention relates to a method to diagnose or determine, or both at the same time, the activity level of autoimmune diseases, the said method comprising a) to supply a biological simple; b) to determine the TßRII-A, TßRII-B and TßRII-Se isoforms level in the biological sample; c) calculate the activity level by performing the quotient between the TßRII-Se level and the level of the addition of TßRII-A y TßRII-B. The isoform level can be measured by detecting the polypeptides of said isoforms or the mRNA of the isoforms in the isolated circulating mononuclear blood cells by means of, for instance, RT-qPCR.

### STATE OF THE ART

Rheumatoid arthritis is an autoimmune disease. It is believed that this kind of diseases occur because of abnormalities of the immune response, be it innate or adaptive, and very probably they have genetic or environmental components.

A diagnosis of this kind of diseases is usually difficult, because their symptoms tend to be relatively non-specific. Consequently, no available blood analysis may exclude, with a degree of certainty, the possibility of an autoimmune disease in individuals showing said symptoms. As the best choice, the performance of a battery of trials and a time assessment by a specialized physician is required, in order to establish whether a patient actually has an autoimmune disease.

To date, there is no definite diagnostic trial confirming a RA diagnosis. Some tests can furnish subjective data that may increase diagnostic certainty and allow the follow up of the disease diagnosis.

Clinical methods currently used for the assessment and classification to assess rheumatoid arthritis (RA), both in clinical trials and in clinical practice, are not sensitive or reproducible enough. The clinical assessment method most frequently utilized is the DAS28, which has potential errors, among which are included the subjectivity of a clinical assessment, the presence of important changes in the joint count with poor repercussion on VSG levels, this marker having a significant weight within the score and the significant differences found in the qualification different observers make of a same patient.

At the present time there exist methods having higher sensitivity and specificity for the detection of joint inflammation, such as NMR and joint ultrasonography, the former being the most sensitive, but also being the most expensive and the one that requires most time for its implementation though, this rendering it a scarcely profitable technique for the periodical assessment needed in clinical practice. On the other hand, high-definition ultrasonography has an accuracy similar to resonance's for the identification of certain pathological changes. However there is no consensus about an echographic scale. Doubts exist about the reliability of fixed images acquisition and interpretation and there is no consensus regarding the assessment system for synovites and there do not exist compound scores using echographic evaluation as a part of patient assessment, to replace joint counts.

Argentine patent application P20130104170 (AR093466A19) discloses the detection of TßRII-A y TßRII-B isoforms in monocytes.

Assays that improve sensitivity and facilitate clinical assessments, in order to make therapeutic decisions, are needed.

### SUMMARY OF THE INVENTION

A method to diagnose or determine, or both at the same time, the activity level of autoimmune diseases is provided, the said method comprising:

a) providing a biological sample;

b) determining the level of the TßRII-A, TßRII-B y TßRII-Se isoforms in that biological sample

c) calculating the activity level by performing the quotient between the TßRII-Se level and the level of the addition of TßRII-A and TßRII-B.

The biological sample may be isolated mononuclear circulating blood cells or the polypeptides of TßRII-A, TßRII-B and TßRII-Se isoforms. The level of isoforms can be measured by the detection of the polypeptides of said isoforms or the mRNA of the isoforms in the isolated circulating mononuclear blood cells. In a preferred embodiment, the level of mRNA in the isoforms is detected by means of RT-qPCR.

In the method the level of the polypeptides of the TßRII-A, TßRII-B and TßRII-Se isoforms can be measured, for example, by measuring the level of polypeptides in the sequences SEQ ID No 8, SEQ ID No 9 and SEQ ID No 10 or in sequences having an identity of 90% at the least.

The present method can be used to diagnose or determine, or do both things at the same time with, the activity level of autoimmune diseases such as rheumatoid arthritis, systemic lupus erythematosus, systemic sclerosis, polydermatomyositis, vasculitis and seronegative spondyloarthropathies.

A kit is provided to diagnose or determine, or do both things at the same time, the activity level of autoimmune diseases. The said kit comprises:

i) specific primers to amplify the TßRII-A, TßRII-B and TßRII-Se isoforms,

ii) reverse transcriptase and Taq polymerase enzymes

iii) means to reveal said isoforms.

The kit may comprise means for the obtainment and isolation of blood mononuclear cells and means of revelation such as, for example, fluorescence, colorimetry and luminiscence.

A method to determine the activity level of autoimmune diseases is provided, the said method comprising

a) providing a biological sample; and

b) determining the level of the TßRII-Se isoform in that biological sample, wherein the activity level of the autoimmune disease is proportional to the ΔCt of TβRII-Se isoform, or the amount of mRNA of TβRII-Se isoform, or the amount of circulating TβRII-Se isoform. For example, the activity of the disease is directly proportional to the quantity of the: circulating TβRII-Se isoform or mRNA of TβRII-Se isoform and indirectly proportional of the ΔCt of TβRII-Se isoform. The level of the TßRII-Se isoform from the biological sample is the amount of mRNA the isoform, or the amount of circulating isoform, or the ΔCt of TβRII-Se isoform and the biological sample is isolated circulating blood cells, preferably isolated mononuclear circulating blood cells.

A method to determine the activity level of autoimmune diseases is provided, the said method comprising:

a) providing a biological sample; and

b) determining the level of the TßRII-A, TßRII-B and TßRII-Se isoforms in that biological sample, wherein the activity of the autoimmune disease is proportional to the sum of the level of TßRII-A, TßRII-B and TßRII-Se isoforms. The level of the said isoforms from the biological sample is the amount of mRNA the isoforms, or the amount of circulating isoforms, or the ΔCt of said isoforms and the biological sample is isolated circulating blood cells, preferably isolated mononuclear circulating blood cells. The activity of the disease is directly proportional to the sum of the quantities of the TßRII-A, TßRII-B and TßRII-Se isoforms and indirectly proportional to sum of ΔCt of the TßRII-A, TßRII-B and TßRII-Se isoforms.

A method to determine the activity level of autoimmune diseases is provided, the method comprising:

a) providing a biological sample; and

b) determining the level of the TßRII-A, TßRII-B and TßRII-Se isoforms in that biological sample, wherein the activity of the autoimmune disease is proportional to the sum of TßRII-A and TßRII-B isoforms, the sum of TßRII-A and TßRII-Se isoforms or the sum of TßRII-B and TßRII-Se isoforms. The level of the said isoforms from the biological sample is the amount of mRNA the isoforms, or the amount of circulating isoforms, or the ΔCt of said isoforms and the biological sample is isolated circulating blood cells, preferably isolated mononuclear circulating blood cells. The activity of the autoimmune disease is inversely proportional to any of the ΔCt of said isoforms sums, or directly proportional to the sum of the quantities of the TβRII-A and TβRII-Se isoforms, the sum of the quantities of TβRII-B and TβRII-Se isoforms or the sum of the quantities of TβRII-B and TβRII-A isoforms. (see FIGS. 10, 11 and 12).

### DESCRIPTION OF THE FIGURES

FIG. 1 shows flow cytometry diagrams that represent populations of monocytes and lymphocytes isolated from the volunteer identified as AR#9. The respective purity percentages are shown;

FIG. 2 shows the graphs for the correlations that are positive between the mRNA level of TßRII-A and TßRII-B in PBMNCs (addition of relative abundance of monocytes and lymphocytes) and clinical determinations of the tender joint count (TJC) and the swollen joint count/SJC). The numbers correspond to the number assigned to each volunteer involved in the study. The analysis of the correlations was carried out by employing Spearman's test, with the use of the OriginPro 8.5.1 software (Origin Lab Corporation, Northampton, Mass., USA). Values of p<0.05 were considered to have statistical significance. rs: Spearman's correlation coefficient;

FIG. 3 shows the graphs for the positive correlation between relative abundances of TßRII-A+TßRII-B mRNA's in PBMNCs, and the DAS28 score. The numbers correspond to the number assigned to each volunteer involved in the study. The analysis of the correlations was carried out by employing Spearman's test, with the use of the OriginPro 8.5.1 software (Origin Lab Corporation, Northampton, Mass., USA). Values of p<0.05 were considered to have statistical significance. rs: Spearman's correlation coefficient;

FIG. 4 shows the positive correlation between the quotient between the relative abundances of TßRII-Se's mRNA and the addition of TßRII-A and TßRII-B in PBMNCs, and the DAS28 score. The numbers correspond to the number assigned to each volunteer involved in the study. The analysis of the correlations was carried out by employing Spearman's test, with the use of the OriginPro 8.5.1 software (Origin Lab Corporation, Northampton, Mass., USA). Values of p<0.05 were considered to have statistical significance. rs: Spearman's correlation coefficient;

FIG. 5 shows (A) Activity of the disease in patients with RA according to DAS28. (B) Activity of the disease according to the quotient between TßRII-Se's mRNA levels and the addition of TßRII-A and TßRII-B levels in PBMNCs (method of present invention) Black dots: high activity according to DAS28. Grey dots: moderate activity according to DAS28. Empty dots: low activity according to DAS28;

FIG. 6 is a schematic depiction of the sites where the primers used in RT-qPCr are adhered. Amplified product TβRII-A (SEQ ID No 1) with primers P3 (SEQ ID No 2) and P2 (SEQ ID No 3): 218 pb. Amplified product TβRII-B (SEQ ID No 4) with primers P1 (SEQ ID No 5) and P2 (SEQ ID No 3): 233 pb. Amplified product TβRII-Se (SEQ ID No 6) with primers P3 (SEQ ID No 2) and P4 (SEQ ID No 7): 137 pb;

FIG. 7 shows a negative correlation between DCt values of TbRII-A in PBMCs, and DAS28-ESR index. Indicating that higher DCt of TbRII-A reflects lower activity; and lower DCt of TbRII-A reflects higher activity. Numbers correspond to the ID number assigned to each volunteer involved in this study. Correlation results were achieved using the Spearman's rank-order correlation test (rₛ: Spearman's rank correlation coefficient), and means were compared using Student's t-test. Both tests were performed using the software OriginPro 8.5.1 (Origin Lab Corporation, Northampton, Mass.). p-values <0.05 were considered statistically significant;

FIG. 8 shows a negative correlation between DCt values of TbRII-B in PBMCs, and DAS28-ESR index. Indicating that higher DCt of TbRII-B reflects lower activity; and lower DCt of TbRII-B reflects higher activity. Numbers correspond to the ID number assigned to each volunteer involved in this study. Correlation results were achieved using the Spearman's rank-order correlation test (rₛ: Spearman's rank correlation coefficient), and means were compared using Student's t-test. Both tests were performed using the software OriginPro 8.5.1 (Origin Lab Corporation, Northampton, Mass.). p-values <0.05 were considered statistically significant;

FIG. 9 shows a negative correlation between DCt values of TbRII-Se in PBMCs, and DAS28-ESR index. Indicating that higher DCt of TbRII-Se reflects lower activity; and lower DCt of TbRII-B reflects higher activity. Numbers correspond to the ID number assigned to each volunteer involved in this study. Correlation results were achieved using the Spearman's rank-order correlation test (rs: Spearman's rank correlation coefficient), and means were compared using Student's t-test. Both tests were performed using the software OriginPro 8.5.1 (Origin Lab Corporation, Northampton, Mass.). p-values <0.05 were considered statistically significant;

FIG. 10 shows a negative correlation between DCt values of TbRII-A+TbRII-B in PBMCs, and DAS28-ESR index. Indicating that higher DCt of TbRII-A+TbRII-B reflects lower activity; and lower DCt of TbRII-A+TbRII-B reflects higher activity. Numbers correspond to the ID number assigned to each volunteer involved in this study. Correlation results were achieved using the Spearman's rank-order correlation test (rₛ: Spearman's rank correlation coefficient), and means were compared using Student's t-test. Both tests were performed using the software OriginPro 8.5.1 (Origin Lab Corporation, Northampton, Mass.). p-values <0.05 were considered statistically significant;

FIG. 11 shows a negative correlation between DCt values of TbRII-A+TbRII-Se in PBMCs, and DAS28-ESR index. Indicating that higher DCt of TbRII-A+TbRII-Se reflects lower activity; and lower DCt of TbRII-A+TbRII-Se reflects higher activity. Numbers correspond to the ID number assigned to each volunteer involved in this study. Correlation results were achieved using the Spearman's rank-order correlation test (rₛ: Spearman's rank correlation coefficient), and means were compared using Student's t-test. Both tests were performed using the software OriginPro 8.5.1 (Origin Lab Corporation, Northampton, Mass.). p-values <0.05 were considered statistically significant;

FIG. 12 shows a negative correlation between DCt values of TbRII-B+TbRII-Se in PBMCs, and DAS28-ESR index. Indicating that higher DCt of TbRII-B+TbRII-Se reflects lower activity; and lower DCt of TbRII-B+TbRII-Se reflects higher activity. Numbers correspond to the ID number assigned to each volunteer involved in this study. Correlation results were achieved using the Spearman's rank-order correlation test (rₛ: Spearman's rank correlation coefficient), and means were compared using Student's t-test. Both tests were performed using the software OriginPro 8.5.1 (Origin Lab Corporation, Northampton, Mass.). p-values <0.05 were considered statistically significant; and

FIG. 13 shows a negative correlation between DCt values of TbRII-A+TbRII-B+TbRII-Se in PBMCs, and DAS28-ESR index. Indicating that higher DCt of TbRII-A+TbRII-B+TbRII-Se reflects lower activity; and lower DCt of TbRII-A+TbRII-B+TbRII-Se reflects higher activity. Numbers correspond to the ID number assigned to each volunteer involved in this study. Correlation results were achieved using the Spearman's rank-order correlation test (rs: Spearman's rank correlation coefficient), and means were compared using Student's t-test. Both tests were performed using the software OriginPro 8.5.1 (Origin Lab Corporation, Northampton, Mass.). p-values <0.05 were considered statistically significant.

### DETAILED DESCRIPTION OF PRESENT INVENTION

For the purposes of the present invention, “RA” are the initials of “rheumatoid arthritis”.

By “addition of relative abundance of monocytes and lymphocytes” it is meant the TßRII-A, TßR IIB or TβRII-Se's mARN level in monocytes and lymphocytes subtracted from the value of the reference known gene.

For the purposes of the present invention the initials PBMNCs are defined as the mononuclear cells (monocytes and lymphocytes) isolated from peripheral blood.

With the term “TßRII-A, TßRIIB or TβRII-Se isoforms”, splicing variants of TGF-ß's Type II receptor that code for the corresponding polypeptides are defined.

This method presents several advantages such as, for example. that it is not necessary to segregate monocytes (plastic adherent cells) from other mononuclear cells (non-adherent to plastic): all of the mononuclear cells can be used, what entails the subsequent reduction of the processing time in 18 hours and without the need for complex equipment (laminar flow bench, CO₂ incubator) or the handling by experienced personnel.

When TßRII-A+TßRII-B are analyzed in all of the mononuclear cells, false positives have occurred that through the inclusion of TßRII-Se, place themselves again within the corresponding activity group according to DAS28. Besides, with the present method better correlations and more significant from a statistical viewpoint have been achieved, than with the previous method.

The present method allows for the identification of different activity levels of rheumatoid arthritis, by means of the quantification in peripheral blood mononuclear cells, of the mRNA of three isoforms of the Type II receptor of TGF-ß. The isoforms are TßRII-A, TßRII-B and TßRII-Se, or the detection of said isoforms as polypeptides, where the TßRII-A sequence is SEQ ID No 8 or sequences having between 90% and 99% of identity; the TßRII-B sequence is SEQ ID No 9 or sequences having between 90% and 99% of identity and the TßRII-Se sequence is SEQ ID No 10 or sequences having between 90% and 99% of identity. In a preferred embodiment, the quantification method is carried out by means of the RT-qPCR technique.

In a preferred embodiment, the present method comprises the obtainment of peripheral blood and a later purification or isolation of the mononuclear cells by means of, for example, density gradient centrifugation (for example, Ficoll-Paque) or by any other known method. For example, known methods are a separation by means of flow cytometry (cell sorting) and magnetic immunoseparation. Afterwards, the isolated cells can be incubated at 37° C. with 5% of CO₂ for 12-16 hours. The adhered cells are the monocytes, while the non-adhered cells are the lymphocytes. Monocyte and lymphocyte (BMNCs) mARN is purified by means of known methods. Finally. The cDNA is generated from the mRNA total of monocytes and lymphocytes and it is quantified by means of PCR in real time (RT-qPCR) with specific primers. Lastly, the corresponding calculations are carried out.

After the adherent (monocytes) and non-adherent (lymphocytes) PBMNCs purification was performed, purity was assessed by means of flow cytometry, use being made of cell granularity (expressed as side scattering of light or SCC) and cell size (expressed as forward scattering of light or FSC). Said measurement varied between 52% and 87% for lymphocytes from volunteers with RA, and between 36% and 89% for monocytes from volunteers with RA (Table 1).

TABLE 1

| ID and sex | Lymphocyte | Monocyte |
|---|---|---|
| of patients | purity (%) | purity (%) |
| 03, F | 51.77 | 58.63 |
| 06, M | 78.96 | 43.31 |
| 09, M | 73.19 | 82.24 |
| 12, F | 74.37 | 67.48 |
| 14, F | 83.80 | 89.35 |
| 08, F | 73.59 | 35.84 |
| 17, F | 86.88 | 81.77 |
| 05, F | 71.49 | 69.15 |

Table 1. Purity of lymphocytes and monocytes isolated from peripheral blood of volunteers having RA established by flow cytometry. The numbers by the volunteers correspond to the ones assigned to said volunteers for the trial.
F; female;
M: male.

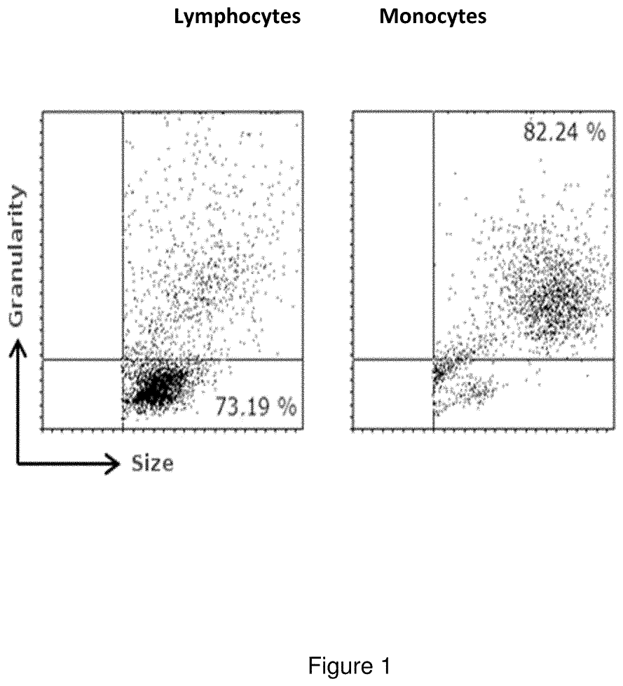

In FIG. 1, representative flow cytometry diagrams and the purity percentage of lymphocyte and monocyte populations are depicted.

With the purpose of assessing the possibility of the development of a method that contribute with objective parameters to determine the activity of AR or other autoimmune diseases, the relative levels of mARN from the diverse isoforms of TβRII where correlated, where the mARN was isolated from PBMNCs (lymphocytes and monocytes) pertaining to RA patients that showed clinical and biochemical parameters of the disease.

Under the light of what is disclosed here. It is understood that it is also possible to employ methods that measure isoform levels as polypeptides that are soluble or are joined to membranes. All of those methods are included within the scope of the present invention,

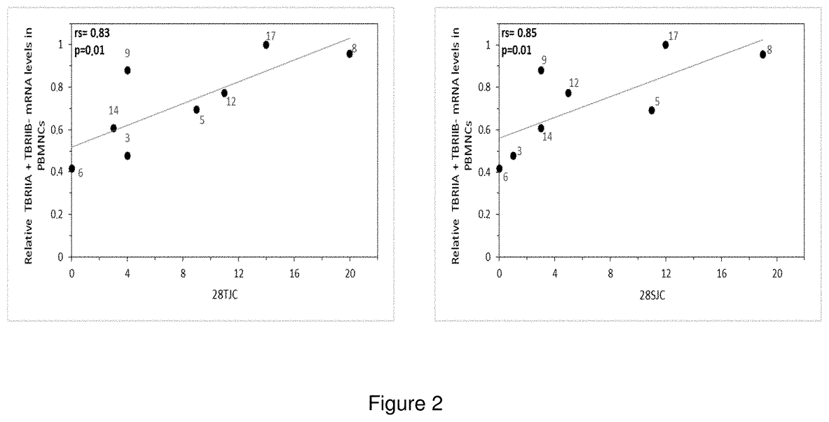

In RA patients it could be seen that their levels of mARN of WI I-A and TβRII-B in PBMNCs are positively, and in a significant way, correlated with the quantity of the tender joints and the quantity of swollen joints (FIG. 2).

The three-parameter DAS28 equation is an index routinely used to determine RA activity and includes the quantity of tender joints (TJC), the quantity of swollen joints (SJC)—both determined in a total quantity of 28 defined joints—and as (SJC)—ambas determinadas sobre un total de 28 articulaciones definidas—, and the erythrocyte sedimentation rate (ESR).

DAS28=0.56√NAD+0.28√NAI+0.70 (ln ESR)

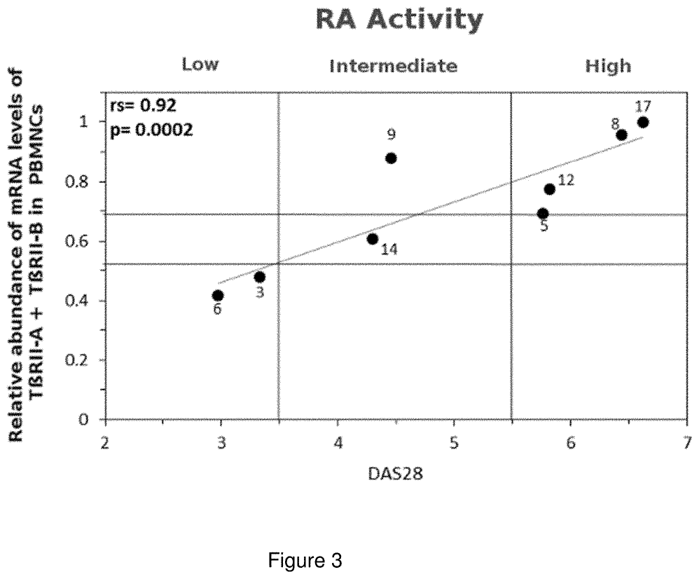

It can be expected that if the correlations between the mRNA levels of TβRII-A and TβRII-B in both populations and the number of tender and swollen joints are significant, the same happens with those between the same mRNA and DAS28, since both of these clinical determinations are contained in the definition of DAS28. It is important to remark that the ESR is also contained in the DAS28 score, but this is a non specific variable that may be affected by other factors, not only from swelling. Because of this, the ESR is generally used together with other determinations of the disease. As expected, DAS28 values correlate positively with mRNA values of both isoforms of the receptor in PBMNCs (TβRII-A and TβRII-B) (FIG. 3).

By using the DAS28 score, three degrees of disease activity can be determined: low (values lower than or equal to 3.5), moderate (values higher than 3.5 and lower than, or equal to, 5.5) and high (values higher than 5.5) (Table 2).

TABLE 2

| | Value | Patient No |
|---|---|---|
| RA Activity | DAS28 | (value DAS28) |
| Low | ≤3.5 | 06 (2.97) |
| | | 03 (3.33) |
| Moderate | >3.5 and | 09 (4.46) |
| | ≤5.5 | 14 (4.30) |
| High | >5.5 | 05 (5.76) |
| | | 12 (5.82) |
| | | 08 (6.44) |
| | | 17 (6.62) |

Table 2. Classification criterion for RA activity according to DAS28. Here are included the patients involved in the trial in the corresponding category of RA according to their value of DAS28.

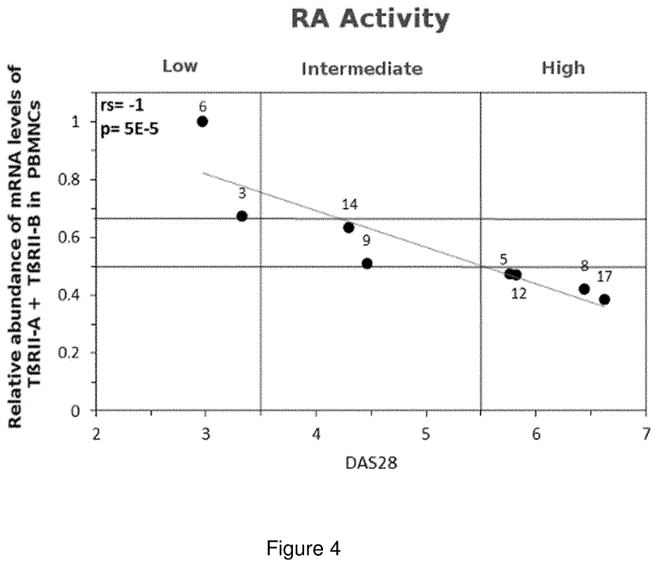

When the significant positive correlations obtained were analyzed in detail, it was seen that the mRNA level of TβRII-A and TβRII-B in PBMNcs from patients with RA is distributed in a greatly similar way in the three groups the disease is classified in according to DAS28. The mRNA levels of TβRII-A and TβRII-B in the PBMNCs from RA patients increase accordingly to the activity increase of the disease, there being a false positive occurrence corresponding to patient 9. However, when the quotient between the relative levels of TβRII-Se and the addition of the two remaining isoforms TβRII-A and TβRII-B (FIG. 4) are included in the calculation, it is obrained a higher negative correlation ((rs=−1) and also more significant from the statistical viewpoint (p=5.5×10⁻⁵) than the one obtained using only the relative abundance of mRNA of TßRIIA plus TßRII-B, where patient 9 is positioned in the group of intermediate activity, in coincidence with the value of DAS28.

Therefore, from the aforesaid analysis the mRNA levels of the three isoforms of TβRII in the PBMNcs from RA patients as candidates to be assessed as new biomarkers for the disease, where selected. It could be determined that the quotient between the mRNA levels of TßRII-Se and the addition of the TßRII-A and TßRII-B levels in PBMNCs from patients with RA diminishes as the disease activity increases and they become distributed in three groups that coincide with the disease categories determined by the DAS28 values.

Then, cut-off values for the levels can be established in the result of the quotient between the mRNA values of TßRII-Se and the addition of mRNA levels of TßRII-A and TßRII-B, to employ this parameter as a biomarker for RA diagnosis, activity determination and prognosis.

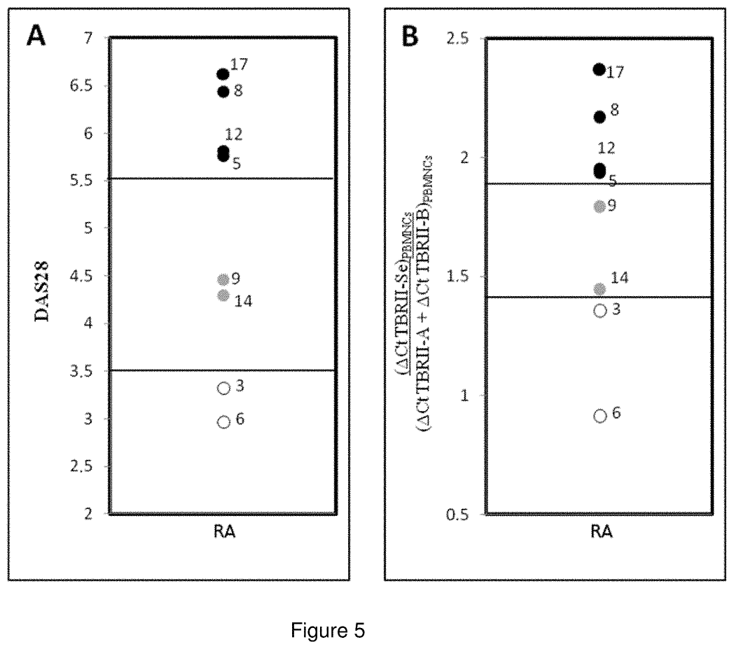

The detection method for RA can be seen in FIG. 5. In a preferred embodiment, the method is based on the use of the Ct parameter (cycle threshold) obtained by RT-qPCR, where ΔCt is the difference of Ct between the value gotten with each one of the isoforms and the Ct of the reference gene, in this case GAPDH. Therefore, the calculation used to perform this approximation is the quotient between ΔCt_{TβRII-Se}/(ΔCt_{TβRII-A}+ΔCt_{TβRII-B}), being ΔCt_{TβRII-Se}=Ct_{TβRII-Se}−Ct_{GAPDH}; ΔCt_{TβRII-A}=Ct_{TβRII-A}−Ct_{GAPDH}; ΔCt_{TβRII-B}=Ct_{TβRII-B}−Ct_{GAPDH}, respectively

In the example, GAPDH has been used as a reference gene, but the experts in this art know that in order to carry out the method of the present invention, any known reference gene may be used.

Thus, it can be established that patients having an isoform quotient higher than 1.8 could be considered as having a high activity of the disease; between 1.8 and 1.4, intermediate activity and below 1.4, low activity.

These indexes are mandatory for the general practitioner, taking into account that they show the degree of activity of the disease, thus permitting the making of therapeutic decisions as a function of objective evidence. It is also possible to monitor the therapeutic response and establish an evolution prognosis. The method disclosed by the present invention clearly is an objective method.

The method according to the present invention allows for the determination of rheumatoid arthritis from, for example, the detection of the levels of messenger RNA, but it is also possible from the measurement of the three codified isoforms of the TGF-ß type II receptor. Because of the high correlation between the transcription of genes of interest and the degree of progress of the pathology, the present invention proposes an efficient and reliable method for the diagnosis and follow-up of the disease.

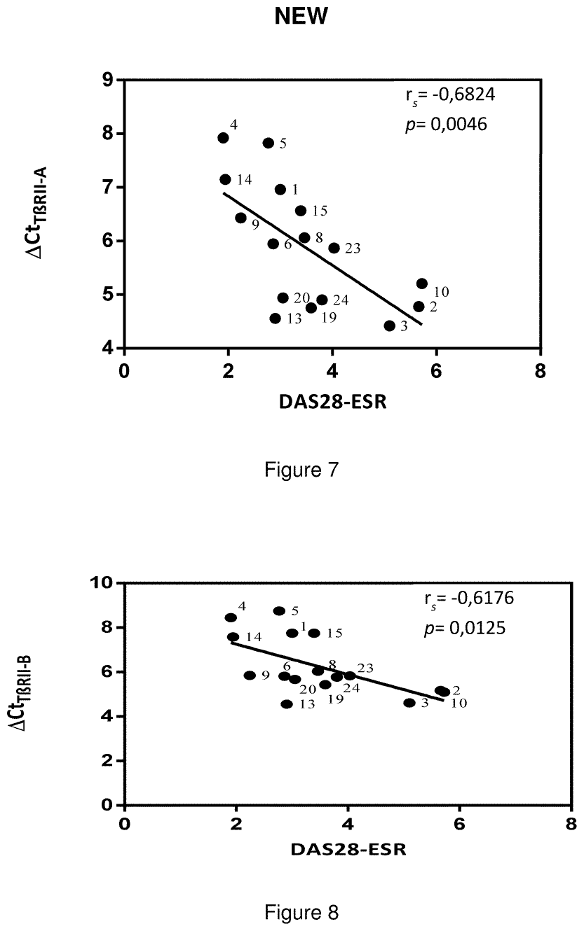

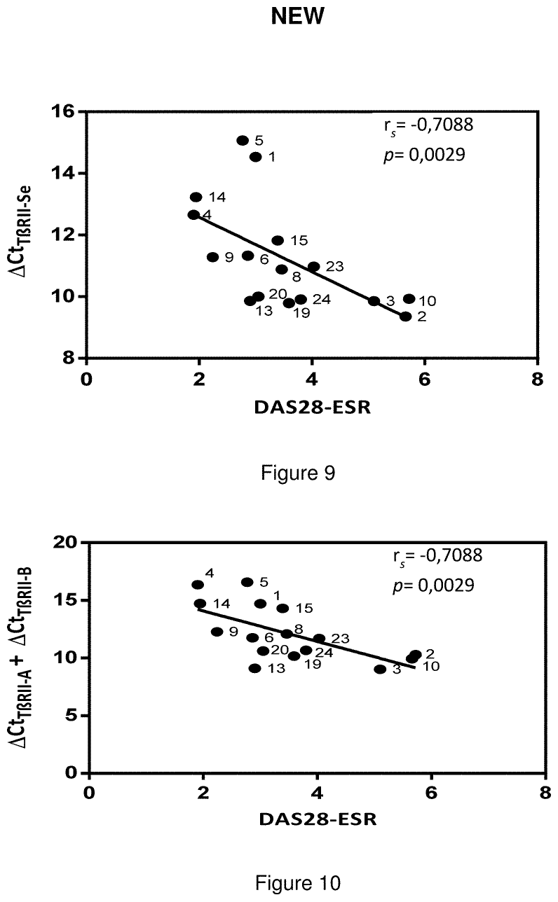

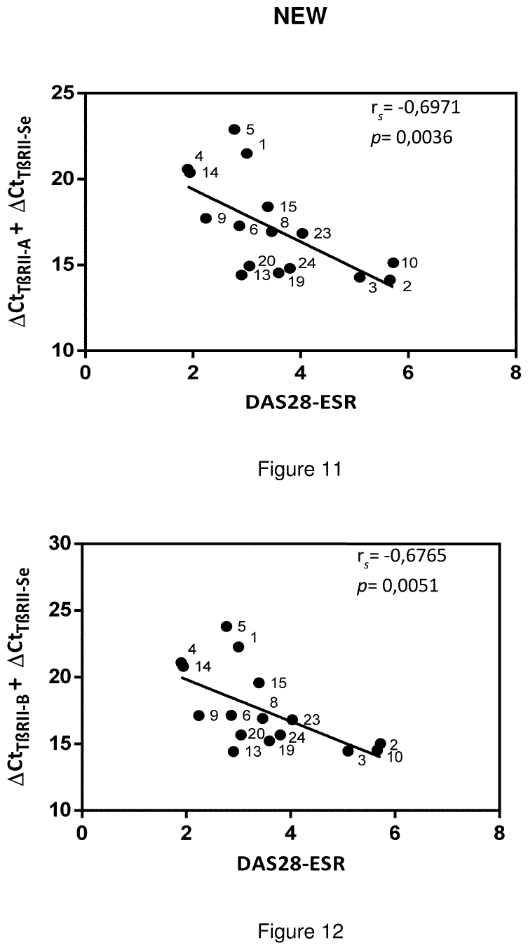

Analyzing the ΔCt of each splice variants individually, we observed that the best correlation with RA disease activity was obtained with TβRII-SE (FIG. 9), followed by the correlation with TβRII-A (FIG. 7) and TβRII-B (FIG. 8).

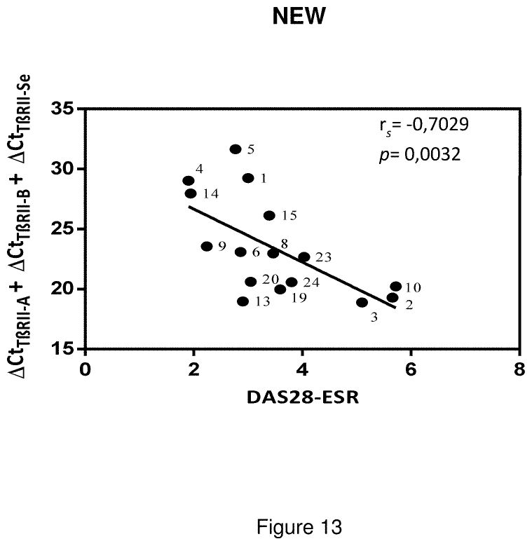

Additionally, a similar correlation with RA disease activity was obtained when the ΔCt of TβRII-SE was added to the ΔCt of TβRII-A and the ΔCt of TβRII-B (FIG. 13).

The present method permits:

The diagnosis and determination of RA activity and other autoimmune diseases, such as systemic lupus erythematosus, systemic sclerosis, polydermatomyositis, vasculitis and seronegative spondyloarthropathies.

To establish the degree of activity of the disease by means of an objective method.

To establish prognoses.

A marker of treatment effectiveness.

The following up of the disease.

The use of quantification methods such as, for example RT-PCR

The diagnosis is made based on peripheral blood samples.

The Ct value determined by RT-qPCR is the cycle number at which the fluorescence signal crossed the threshold line. ΔCt values are those where the Ct value of each splice variant is normalized to a reference gene, (giving rise to the following ΔCt values: ΔCt TβRII-A=(Ct_{TβRII-A}−Ct_{GAPDH}), ΔCt TβRII-B=(Ct_{TβRII-B}−Ct_{GAPDH}), and ΔCt TβRII-Se=(Ct_{TβRII-Se}−Ct_{GAPDH}).

ΔCt is inversely proportional to the actual amount of mRNA, therefore an expert could also measure the amount of mRNA by other means, and would obtain similar results respect to the autoimmune disease activity.

This invention is best illustrated by the following examples, which should not be construed as a limitation imposed on the scope of the invention: on the contrary, it must be clearly understood that other embodiments, modifications and equivalents of the present invention can be resorted to, because the reading of the present specifications may suggest that to the experts in this art without departing from the spirit or the scope, or both at the same time, of the enclosed claims.

### EXAMPLES

### Example 1: Samples from Patients with Rheumatoid Arthritis (RA)

The samples were taken at the Instituto Médico CER, Quilmes, Buenos Aires Province, Argentina, and the clinical and biochemical studies were performed at that same Institution. All of the volunteers with RA (N=8) included in this study signed an informed consent prior to the taking of data and samples. The trial was approved by the bioethics institutional commission of the Servicio de Reumatologia del Instituto Medico CER, Quilmes, Buenos Aires (Rheumatology Department of the aforementioned Instituto CER) and the Health ministry of Buenos Aires Province. The criteria for the inclusion of volunteers in this trial were that those volunteers would be willing to give their previous informed consent, were older than 18 years of age and met the ARA 1987 criteria (Arnett, F. C. et al., Arthritis Rheum. 1988 31(3):315-24. Criteria for exclusion were diseases and their concomitant medication that might generate a bias of the interpretation of results, such as the use of biological drugs.

Information on age, sex, evolution time of the disease, concomitant diseases, medication for RA, concomitant medication was gotten, and the erythrocyte sedimentation rate (ESR) was measured. On diseased volunteers it was determined the number of tender joints, radiological injuries and bone mineral density in lumbar spine and femoral neck. Data from three parameters, DAS28 (Disease Activity Scores using 28 joint counts), HAQ (Health Assessment Questionnaire) and VAS (Visual Analogue Scale) were also obtained.

The data and clinical and biochemical determinations of the volunteers are summarized in the Table 3, below:

TABLE 3

| Characteristics | RA (n = 8) | |
|---|---|---|
| Demographic | | |
| Age, years | 50.2 | (23-78) |
| Sex F/M | 6/2 | |
| Duration of disease, years | 11.58 | (0.25-21) |
| Clinical determinations | | |
| Number of tender joints | 9.2 | (0-20) |
| Number of swollen joints | 7.6 | (0-19) |
| DAS28 | 4.77 | (2.58-6.65) |
| HAQ | 1.102 | (0.125-2800) |
| VAS pain in patient, mm | 43.6 | (20-72) |
| VAS activity of patient, mm | 43.6 | (29-78) |
| VAS physician, mm | 47 | (5-80) |
| Laboratory analytical parameter | | |
| ESD, mm | 36.3 | (9-65) |
| Treatment with drugs | | |

| | |
|---|---|
| NSAID | 5/8 |
| DMARD | 6/8 |

Table 3. The values correspond to means and, inside parentheses, minimums and maximums.
F: female;
M: male;
ESD: erytho cell sedimentation;
NSAID: , Non-Steroidal Anti-Inflammatory Drugs;
DMARD: Disease-Modifying Anti-Rheumatic Drugs;

### Example 2: Isolation of Leukocytes and Separation of the Total of Lymphocytes, and of Monocytes

From heparinized peripheral blood, a centrifugation per gradient was carried out with Ficoll-Paque™ PLUS (GE Healthcare Bio-Sciences AB), after which a fraction containing peripheral blood mononuclear cells (PBMCs) was obtained. The number of PBMCs was quantified in A Neubauer chamber by means of an exclusion method with trypan blue.

The total quantity of lymphocytes and the monocytes of patients with RA were separated from the PBMCs on the basis of their differential properties of adherence to plastic. In order to do that, the PBMCs were cultured for 2 hours in RPMI medium supplemented with 10% of human serum (HS), after which the cell supernatant containing lymphocytes mainly, was collected. The monocytes, adhered to the culture vial, were grown for 16 hours, after which time they were collected by means of treatment with trypsin-EDTA. The purity of both populations was determined by flow cytometry with a BD FACSCalibur™ (BD, USA equipment), use being made of cell size (FSC) and cell complexity (SSC). The viable cells from both populations were quantified by the method of exclusion with trypan blue in a Neubauer chamber. They were kept at −80° C. in lisis buffer for the extraction of RNA, until they were used.

### Example 3: Flow Cytometry Assay

For the analysis by flow cytometry, a minimum of 5×10⁴ quantified viable cells were employed in a Neubauer chamber by the exclusion method with trypan blue. The cells were re-suspended in 100 μl of fixing solution. The measurements were performed with a FACSCalibur (BD Biosciences, USA) equipment and the analysis of the data obtained was done with the WinMDI program.

### Example 4: Quantitative RT-PCR (RT-qPCR)

Extraction of RNA. The RNA was isolated from 10⁴-10⁶ cells, to do which the commercial kit Absolutely RNA® Miniprep Kit (Stratagene, La Jolla, Calif., USA) or the commercial kit SV Total RNA Isolation System (Promega, Wis., USA), were used, the instructions from the manufacturers having been followed in each case. The purified RNA was re-suspended in 50 μl of elution buffer or nuclease-free water, according to the recommendations of the manufacturer of each kit, and it was quantified, a spectrophotometer BioPhotometer (Eppendorf, Germany) having been used, and it was kept at −80° C. until the moment to use it.

Generation of complementary DNA (cDNA). Reverse transcription reactions were carried out in a final volume of 40 μl composed of 1.5 μg of RNA, 0.2 μM of primers OligodT15 (Promega, Wis., USA) and 200 U of reverse transcriptase from M-MLV (Promega, Wis., USA), the manufacturer's instructions having been followed. The cDNA generated was kept at −80° C. until its use.

RT-qPCR reactions were carried out using a Mx3005P™ (Stratagene, USA) equipment. The reactions were performed in a final volume of 10 μl, composed of 2 μl of cDNA, Fast Start Universal SYBR Green Master (ROX) 1× (Roche Applied Science, Mannheim, Germany), 300 nM of direct and reverse primers for the amplification of TβRII-A, TβRII-B and GAPDH[P3 (SEQ ID No 2) and P2 (SEQ ID No 3), P1 (SEQ ID No 5) and P2 (SEQ ID No 3) and GAPDH-F (SEQ ID No 11) and GAPDH-R (SEQ ID No 12)] and 200 nM for the amplification of TβRII-Se [P3 (SEQ ID No 2) and P4 (SEQ ID No 7)]. In each assay the samples were analyzed in triplicate.

As a control normalizing gene, primers that amplify the constitutive gene of Glyceraldehyde 3-phosphate dehydrogenase (GAPDH), the sequences of which are: GAPDH-F: 5′ GTCAGTGGTGGACCTGACC 3′ (SEQ ID No 11), GAPDH-R: 5′ TGAGCTTGACAAAGTGGTCG 3′ (SEQ ID No 12), were utilized.

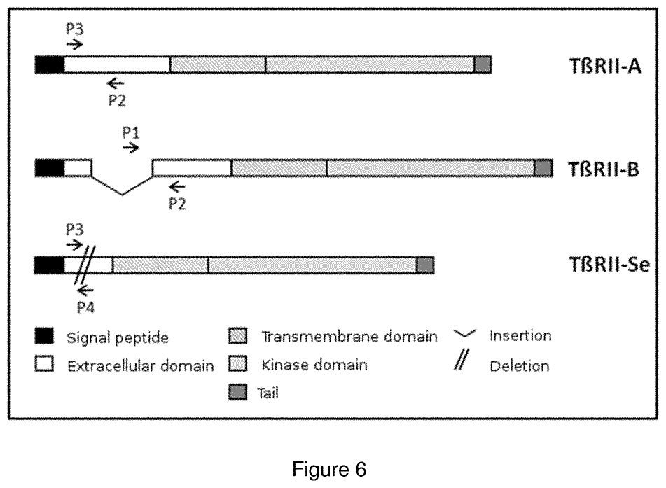

The conditions for amplification were: 1 cycle of 15 minutes at 95° C. and 40 cycles of 10 seconds at 95° C. and 30 seconds at 62° C. Afterwards, a dissociation curve to assess the presence of nonspecific amplification products was carried out. The sequences of the primers employed were: P1: 5′CTGTAATAGGACTGCCCATC 3′(SEQ ID No 5), P2: 5′ TCTCTAGTGTTATGTTCTCGTC 3′ (SEQ ID No 3), P3: 5′ GCACGTTCAGAAGTCGGTTAA 3′ (SEQ ID No 2), P4: 5′ GCACTTTGGAGAAGCAGGATT 3′ (SEQ ID No 7). For the specific amplification of the different isoforms of TβRII, diverse combinations of these primers were used. For TβRII-A, the primers P2 and P3 were used; for TβRII-B, P1 and P2 and for TβRII-Se, the primers P3 and P4 (see FIG. 6).

Statistics: The statistical analysis of the data coming from the RT-qPCR's was performed by means of the InfoStat version 2010 software (Di Rienzo, J. A. et al., http://www.infostat.com.ar).

The analysis of the correlations was performed through Spearman's test. To carry out these tests the OriginPro 8.5.1 (Origin Lab Corporation, Northampton, Mass., USA software was utilized. In all of the cases, values of p<0.05 were considered statistically significant.

### Example 5: New Tests

### Volunteers and Samples

Peripheral blood was collected by venipuncture from 16 RA patients. Patients were diagnosed according to the ACR/EULAR 2010 criteria (Aletaha et al., 2010). All procedures were approved by CER Medical Institute Research Ethics Committee, and the Comisión Conjunta de Investigación en Salud, Department of Health, Buenos Aires Province, Argentina, registered under the number 2919/653/13. Procedures were performed after signing off a voluntary informed consent, by the donors. Exclusion criteria included severe anaemia, autoimmune diseases different from RA, any other disease/condition able to increase ESR, treatment with biological drugs, treatment with disease-modifying anti-rheumatic drugs (DMARDs) except methotrexate, and with drugs with known effect on the TGF-β signalling cascade (losartan).

### Clinical and Laboratory Data

Clinical determinations included swollen and tender joints, Health Assessment Questionnaire Disability Index (HAQ-DI), and Visual Analogue Scale (VAS) scores, such as physician global VAS, patient global VAS, and pain VAS. ESR was determined by the Westergren Method (Expert Panel on Blood Rheology, 1993). Disease activity of RA was determined by the 28 joint count disease activity score, including ESR values, or DAS28-ESR with three variables (Prevoo et al., 1995). Categories of disease activity based on the DAS28-ESR were defined as follows: remission=DAS28≤2.4, low=2.4<DAS28≤3.6, moderate=3.6<DAS28≤5.5, high=DAS28>5.5 (Aletaha et al., 2005). Clinical determinations were assessed by three independent rheumatologist. Additional information regarding the volunteers is listed in Table 4.

### Purification of Different Subpopulations of Leukocytes

Peripheral blood mononuclear cells (PBMCs) were purified by Ficoll-Paque™ PLUS (GE Healthcare Bio-Sciences, Pittsburgh, Pa.) gradient centrifugation, 3-4 hours after venipuncture.

Cell purity was assessed by measuring and plotting Forward Scatter (FSC) versus Side Scatter (SSC) parameters in a FACSCalibur platform (BD Biosciences, San Jose, Calif.).

### RNA Isolation and RT-qPCR

Total RNA from each blood cell population was obtained using the SV Total RNA Isolation System. Total RNA was quantitated by using the RiboGreen RNA quantification Assay (Invitrogen). The presence of RT-qPCR inhibitors, in the RNA samples, was checked by using the qPCR amplification of the SPUD amplicon. cDNA was generated using 0.5 μg RNA, Superscript IV Reverse Transcriptase, and oligo dT₍₂₀₎ primers, according to the indications stated by the manufacturer (Invitrogen). mRNA integrity was estimated by the 3′:5′ assay by RT-qPCR using GAPDH primers. To measure the mRNA levels of TGF-β type II receptor splice variants (TβRII-A and TβRII-B and TβRII-Se), qPCR was performed in a StepOne real-time PCR System (Applied Biosystems). Reactions, in triplicates, were performed in 10 μl containing 2 μl of cDNA (dilution 1/15), 300 nM of the corresponding Forward and Reverse primers, and Power SYBR Green Master Mix (Applied Biosystems).

The qPCR amplification conditions were: 10 minutes at 95° C., 1 cycle, and 40 cycles of 15 seconds at 95° C. and 60 seconds at 60° C. Subsequently, a melting curve was performed to check amplification specificity. The sequence of the primers used was as follows:

P1:
(SEQ ID No 5)
5′ CTGTAATAGGACTGCCCATC 3′
P2:
(SEQ ID No 3)
5′ TCTCTAGTGTTATGTTCTCGTC 3′
P3:
(SEQ ID No 2)
5′ GCACGTTCAGAAGTCGGTTAA 3′
P4:
(SEQ ID No 7)
5′ GCACTTTGGAGAAGCAGGATT 3′

For the specific amplification of each TβRII splice variant different primer combination was used: for TβRII-A primers P2 and P3, for TβRII-B primers P1 and P2, and for TβRII-Se primers P3 y P4. After RT-qPCR, we obtained three cycle threshold (Ct) values: Ct_{TβRII-A}, Ct_{TβRII-B}, and Ct_{TβRII-Se}.

The Ct value determined by RT-qPCR is the cycle number at which the fluorescence signal crossed the threshold line. These Ct values were normalized to glyceraldehyde-3-phosphate dehydrogenase (GAPDH) mRNA levels, (primer GAPDH forward: 5′ TGCACCACCAACTGCTTAGC 3′ (SEQ ID No 11) Primer GAPDH reverse: 5′ GGCATGGACTGTGGTCATGAG 3′) (SEQ ID No 12) giving rise to the following ΔCt values: ΔCtTβRII-A=(Ct_{TβRII-A}−Ct_{GAPDH}), ΔCt TβRII-B=(Ct_{TβRII-B}−Ct_{GAPDH}), and ΔCtTβRII-Se=(Ct_{TβRII-Se}−Ct_{GAPDH}).

TABLE 4
Volunteers' baseline characteristics.

| | RA (n = 16) | |
|---|---|---|
| Age (years old) | 48.4 | [27-74] |
| Gender (F/M) | 13/3 | |
| Disease duration (years) | 6.25 | [0.5-29] |
| 28TJC (0-28) | 4.06 | [0-23] |
| 28SJC (0-28) | 1.75 | [0-10] |
| DAS28-ESR | 3.35 | [1.9-5.72] |
| HAQ | 1.1 | [0-2.5] |
| Patient's VAS (mm) | 48.75 | [10-90] |
| Physician's VAS (mm) | 37.5 | [10-90] |
| Global VAS (mm) | 41.85 | [10-90] |
| ESR (mm/hr) | 26.75 | [5-63] |
| Drug treatments | | |

| | |
|---|---|
| NSAIDs | 3/16 |
| DMARDs | 16/16 |

Values are expressed as means and, in square brackets, minimum and maximum values.

SEQUENCE LIST

| | |
|---|---|
| <110> | Conicet, Articular Fundation, and San Martin University |
| <120> | METHOD FOR DETERMINING THE ACTIVITY OF AUTOIMMUNE DISEASES |
| | AND KIT |
| <130> | Dr Dewey et. al., |
| <140> | US 16/349,381 |
| <141> | 2019 May 13 |
| <160> | 12 |
| <170> | PatentIn version 3.5 |
| <210> | 1 |
| <211> | 218 |
| <212> | DNA |
| <213> | Homo sapiens |
| <400> | 1 |

| | |
|---|---|
| gcacgttcag aagtcggtta ataacgacat gatagtcact gacaacaacg gtgcagtcaa | 60 |
| gtttccacaa ctgtgtaaat tttgtgatgt gagattttcc acctgtgaca accagaaatc | 120 |
| ctgcatgagc aactgcagca tcacctccat ctgtgagaag ccacaggaag tctgtgtggc | 180 |
| tgtatggaga aagaatgacg agaacataac actagaga | 218 |

| | |
|---|---|
| <210> | 2 |
| <211> | 21 |
| <212> | DNA |
| <213> | Artificial Sequence |
| <220> | |
| <223> | P3 |
| <400> | 2 |

| | |
|---|---|
| gcacgttcag aagtcggtta a | 21 |

| | |
|---|---|
| <210> | 3 |
| <211> | 22 |
| <212> | DNA |
| <213> | Artificial Sequence |
| <220> | |
| <223> | P2 |
| <400> | 3 |

| | |
|---|---|
| tctctagtgt tatgttctcg tc | 22 |

| | |
|---|---|
| <210> | 4 |
| <211> | 233 |
| <212> | DNA |
| <213> | Homo sapiens |
| <400> | 4 |

| | |
|---|---|
| ctgtaatagg actgcccatc cactgagaca tattaataac gacatgatag tcactgacaa | 60 |
| caacggtgca gtcaagtttc cacaactgtg taaattttgt gatgtgagat tttccacctg | 120 |
| tgacaaccag aaatcctgca tgagcaactg cagcatcacc tccatctgtg agaagccaca | 180 |
| ggaagtctgt gtggctgtat ggagaaagaa tgacgagaac ataacactag aga | 233 |

| | |
|---|---|
| <210> | 5 |
| <211> | 20 |
| <212> | DNA |
| <213> | Artificial Sequence |
| <220> | |
| <223> | P1 |
| <400> | 5 |

| | |
|---|---|
| ctgtaatagg actgcccatc | 20 |

| | |
|---|---|
| <210> | 6 |
| <211> | 137 |
| <212> | DNA |
| <213> | Homo sapiens |
| <400> | 6 |

| | |
|---|---|
| gcacgttcag aagtcggtta ataacgacat gatagtcact gacaacaacg gtgcagtcaa | 60 |
| gtttccacaa ctgtgtaaat tttgtgatgt gagattttcc acctgtgaca accagaaatc | 120 |
| ctgcttctcc aaagtgc | 137 |

| | |
|---|---|
| <210> | 7 |
| <211> | 21 |
| <212> | DNA |
| <213> | Artificial Sequence |
| <220> | |
| <223> | P4 |
| <400> | 7 |

| | |
|---|---|
| gcactttgga gaagcaggat t | 21 |

| | |
|---|---|
| <210> | 8 |
| <211> | 167 |
| <212> | PRT |
| <213> | Homo sapiens |
| <400> | 8 |

Met Gly Arg Gly Leu Leu Arg Gly Leu Trp Pro Leu His Ile Val Leu
1 5 10 15
Trp Thr Arg Ile Ala Ser Thr Ile Pro Pro His Val Gln Lys Ser Val
20 25 30
Asn Asn Asp Met Ile Val Thr Asp Asn Asn Gly Ala Val Lys Phe Pro
35 40 45
Gln Leu Cys Lys Phe Cys Asp Val Arg Phe Ser Thr Cys Asp Asn Gln
50 55 60
Lys Ser Cys Met Ser Asn Cys Ser Ile Thr Ser Ile Cys Glu Lys Pro
65 70 75 80
Gln Glu Val Cys Val Ala Val Trp Arg Lys Asn Asp Glu Asn Ile Thr
85 90 95
Leu Glu Thr Val Cys His Asp Pro Lys Leu Pro Tyr His Asp Phe Ile
100 105 110
Leu Glu Asp Ala Ala Ser Pro Lys Cys Ile Met Lys Glu Lys Lys Lys
115 120 125
Pro Gly Glu Thr Phe Phe Met Cys Ser Cys Ser Ser Asp Glu Cys Asn
130 135 140
Asp Asn Ile Ile Phe Ser Glu Glu Tyr Asn Thr Ser Asn Pro Asp Leu
145 150 155 160
Leu Leu Val Ile Phe Gln Val
165

| | |
|---|---|
| <210> | 9 |
| <211> | 192 |
| <212> | PRT |
| <213> | Homo sapiens |
| <400> | 9 |

Met Gly Arg Gly Leu Leu Arg Gly Leu Trp Pro Leu His Ile Val Leu
1 5 10 15
Trp Thr Arg Ile Ala Ser Thr Ile Pro Pro His Val Gln Lys Ser Asp
20 25 30
Val Glu Met Glu Ala Gln Lys Asp Glu Ile Ile Cys Pro Ser Cys Asn
35 40 45
Arg Thr Ala His Pro Leu Arg His Ile Asn Asn Asp Met Ile Val Thr
50 55 60
Asp Asn Asn Gly Ala Val Lys Phe Pro Gln Leu Cys Lys Phe Cys Asp
65 70 75 80
Val Arg Phe Ser Thr Cys Asp Asn Gln Lys Ser Cys Met Ser Asn Cys
85 90 95
Ser Ile Thr Ser Ile Cys Glu Lys Pro Gln Glu Val Cys Val Ala Val
100 105 110
Trp Arg Lys Asn Asp Glu Asn Ile Thr Leu Glu Thr Val Cys His Asp
115 120 125
Pro Lys Leu Pro Tyr His Asp Phe Ile Leu Glu Asp Ala Ala Ser Pro
130 135 140
Lys Cys Ile Met Lys Glu Lys Lys Lys Pro Gly Glu Thr Phe Phe Met
145 150 155 160
Cys Ser Cys Ser Ser Asp Glu Cys Asn Asp Asn Ile Ile Phe Ser Glu
165 170 175
Glu Tyr Asn Thr Ser Asn Pro Asp Leu Leu Leu Val Ile Phe Gln Val
180 185 190

| | |
|---|---|
| <210> | 10 |
| <211> | 80 |
| <212> | PRT |
| <213> | Homo sapiens |
| <400> | 10 |

Met Gly Arg Gly Leu Leu Arg Gly Leu Trp Pro Leu His Ile Val Leu
1 5 10 15
Trp Thr Arg Ile Ala Ser Thr Ile Pro Pro His Val Gln Lys Ser Val
20 25 30
Asn Asn Asp Met Ile Val Thr Asp Asn Asn Gly Ala Val Lys Phe Pro
35 40 45
Gln Leu Cys Lys Phe Cys Asp Val Arg Phe Ser Thr Cys Asp Asn Gln
50 55 60
Lys Ser Cys Phe Ser Lys Val His Tyr Glu Gly Lys Lys Lys Ala Trp
65 70 75 80

| | |
|---|---|
| <210> | 11 |
| <211> | 19 |
| <212> | DNA |
| <213> | Artificial Sequence |
| <220> | |
| <223> | GAPDH-F |
| <400> | 11 |

| | |
|---|---|
| gtcagtggtg gacctgacc | 19 |

| | |
|---|---|
| <210> | 12 |
| <211> | 20 |
| <212> | DNA |
| <213> | Artificial Sequence |
| <220> | |
| <223> | GAPDH-R |
| <400> | 12 |

| | |
|---|---|
| tgagcttgac aaagtggtcg | 20 |

1. A method to determine the activity level of autoimmune diseases, the method comprising the steps of:
a) providing a biological sample;
b) determining the level of the TßRII-A, TßRII-B y TßRII-Se isoforms in the biological sample; and
c) calculating the activity level by performing the quotient between the TßRII-Se level and the level of the addition of TßRII-A to TßRII-B.

2. The method according to claim 1, wherein the said biological sample comprises isolated mononuclear circulating blood cells.

3. The method according to claim 1, wherein the said biological sample comprises the circulating TßRII-A, TßRII-B and TßRII-Se isoforms.

4. The method according to claim 1, wherein the level of the TßRII-A, TßRII-B and TßRII-Se isoforms from the biological sample comprises the mRNA level of said isoforms.

5. The method according to claim 1, wherein the level of the TßRII-A, TßRII-B and TßRII-Se isoforms from the biological sample comprises the polypeptide level of the sequences SEQ ID No 8, SEQ ID No 9 and SEQ ID No 10 or sequences having at least a 90% of identity with said sequences.

6. The method according to claim 1, wherein the step b) is carried out by RT-qPCR.

7. The method according to claim 1, wherein the autoimmune disease is selected from the group consisting of rheumatoid arthritis, systemic lupus erythematosus, systemic sclerosis, polydermatomyositis, vasculitis, and seronegative spondyloarthropathies.

8. A kit to determine the activity level of autoimmune diseases, comprising:
i) specific primers to amplify the TßRII-A, TßRII-B y TßRII-Se isoforms,
ii) reverse transcriptase and Taq polymerase enzymes, and
iii) a media to reveal said isoforms.

9. The kit according to claim 8, further including a media for the obtainment and isolation of blood mononuclear cells.

10. The kit according to claim 8, wherein the autoimmune disease is selected from the group consisting of rheumatoid arthritis, systemic lupus erythematosus, systemic sclerosis, polydermatomyositis, vasculitis, and seronegative spondyloarthropathies.

11. The kit according to claim 8, wherein the media to reveal the isoforms is selected from the group consisting of fluorescence, colorimetry, and luminescence.

12. A method to determine the activity level of autoimmune diseases, comprising the steps of:
a) providing a biological sample; and
b) determining the level of a TßRII-Se isoform in the biological sample, wherein an activity level of the autoimmune disease is proportional to the level of TßRII-Se isoform.

13. The method according to claim 12, wherein the level of TßRII-Se isoform is the selected from the group comprising ΔCt of TβRII-Se isoform, amount of mRNA of T□RII-Se isoform, and circulating T□RII-Se isoform.

14. The method according to claim 12, wherein the said biological sample comprises isolated circulating blood cells.

15. The method according to claim 14, wherein the biological sample comprises isolated mononuclear circulating blood cells

16. A method to determine the activity level of autoimmune diseases comprising the steps of:
a) providing a biological sample; and
b) determining a level of TßRII-A, TßRII-B and TßRII-Se isoforms in the biological sample, wherein the activity of the autoimmune disease is proportional to the sum of TßRII-A, TßRII-B and TßRII-Se isoforms.

17. The method according to claim 16, wherein the level of the TßRII-A, TßRII-B and TßRII-Se isoforms from the biological sample is selected from the group comprising ΔCt of said isoforms, amount of mRNA of said isoforms, and circulating isoforms.

18. The method according to claim 16, wherein the biological sample comprises isolated blood cells.

19. The method according to claim 18, wherein the biological sample comprises isolated mononuclear circulating blood cells.

20. A method to determine the activity level of autoimmune diseases comprising the steps of:
a) providing a biological sample; and
b) determining a level of TßRII-A, TßRII-B and TßRII-Se isoforms in the biological sample, wherein the activity of the autoimmune disease is proportional to the group consisting of the sum of level of TßRII-A and TßRII-B isoforms, the sum of level of TßRII-A and TßRII-Se isoforms and the sum of the level of TßRII-B and TßRII-Se isoforms, wherein the activity of the autoimmune disease is proportional to any of said sums.

21. The method according to claim 20, wherein the level of the TßRII-A, TßRII-B and TßRII-Se isoforms from the biological sample is selected from the group comprising ΔCt of said isoforms, amount of mRNA of said isoforms and the circulating isoforms.

22. The method according to claim 20, wherein the biological sample comprises isolated blood cells.

23. The method according to claim 20, wherein the biological sample comprises isolated mononuclear circulating blood cells.
